(19) United States
(12) Patent Application Publication
(10) Pub. No.: US 2026/0232262 A1
(43) Pub. Date: Aug. 13, 2026

(54) PLACEBO RESPONSE PREDICTION FOR PSYCHIATRIC PATIENTS

(71) Applicant: Alto Neuroscience, Inc., Mountain View, CA (US)

(72) Inventors: Amit Etkin, Mountain View, CA (US); Patricio O'Donnell, Mountain View, CA (US); Chao Wang, Mountain View, CA (US); Joshua Jordan, Mountain View, CA (US)

(21) Appl. No.: 19/537,878

(22) Filed: Feb. 12, 2026

Related U.S. Application Data
(60) Provisional application No. 63/757,752, filed on Feb. 12, 2025.

(51) Int. Cl. A61B 5/00; A61B 5/372

(52) U.S. Cl. CPC A61B 5/4848; A61B 5/372; A61B 5/7264

(57) ABSTRACT

This invention relates to methods for predicting treatment non-specific response (such as a placebo effect) in psychiatric patients with electroencephalogram (EEG) measurements, including, e.g., spatially normed alpha (8-12 Hz) power features.

### RELATED APPLICATION

This application claims priority to U.S. Provisional Application Serial Number 63/757,752 (“the ‘752 application”), filed February 12, 2025. The ‘752 application is hereby incorporated by reference in its entirety.

### FIELD OF THE INVENTION

This invention relates to a method for predicting treatment non-specific response (such as a placebo effect) in psychiatric patients with electroencephalogram (EEG) measurements including, for example, spatially normed alpha (8-12 Hz) power features.

### BACKGROUND OF THE INVENTION

In clinical trials for antidepressants, a substantial number of patients show improvement in their symptoms even when given a placebo. This can make it difficult to determine the true efficacy of an experimental drug in a clinical trial. See Rutherford et al., Am J Psychiatry, 2013, 170:723-733.

U.S. Patent Publication No. 2021/0353224 discloses methods for determining whether a subject suffering from depression will respond to treatment with an antidepressant or a placebo.

There is a continuing need for improved methods for conducting clinical trials to properly evaluate clinical drugs and devices and properly weigh or exclude patients who exhibit material placebo effects.

### SUMMARY OF THE INVENTION

The present inventors have discovered that a treatment non-specific response (such as a placebo effect) in a psychiatric patient can be predicted based on a pattern of brain activity as recorded through electroencephalography (EEG), for example, including spatially normed alpha (8-12 Hz) power features. As will be disclosed herein, the use of these spatially normed alpha features (which highlight regional differences in the brain) may be used as part of a novel pipeline for predicting placebo responsiveness in patients.

One aspect of the invention is a method for predicting the response of a patient to a placebo or sham treatment (e.g., a placebo treatment), where the patient suffers from a psychiatric disorder. The method comprises: collecting brain wave activity data via EEG electrodes applied to the patient before administration of the placebo treatment or performing the sham treatment; analyzing the brain wave activity data to calculate one or more EEG measures, and predicting an outcome of the placebo or sham treatment in the patient based on the one or more EEG measures, wherein the one or more EEG measures comprise one or more of (i) delta power features (such as relative delta power features, spatially normed delta power features, or spatially normed relative delta power features), (ii) theta power features (such as relative theta power features, spatially normed theta power features, or spatially normed relative theta power features), (iii) alpha power features (such as relative alpha power features, spatially normed alpha power features, or spatially normed relative alpha power features), (iv) beta power features (such as relative beta power features, spatially normed beta power features, or spatially normed relative beta power features), (v) gamma power features (such as relative gamma power features, spatially normed gamma power features, or spatially normed relative gamma power features), (vi) cordance features in the delta, theta, alpha, beta, or gamma frequency range, (vii) sample entropy in the delta, theta, alpha, beta, or gamma frequency range, (viii) Lempel-Ziv complexity in the delta, theta, alpha, beta, or gamma frequency range, (ix) Lyapunov complexity in the delta, theta, alpha, beta, or gamma frequency range, (x) Katz fractal dimension in the delta, theta, alpha, beta, or gamma frequency range, (xi) covariance in the delta, theta, alpha, beta, or gamma frequency range, (xii) power envelope connectivity in the delta, theta, alpha, beta, or gamma frequency range, (xiii) coherence in the delta, theta, alpha, beta, or gamma frequency range, (xiv) weighted phase lag index in the theta, alpha, beta, or gamma frequency range, (xv) beta/theta ratio, (xvi) beta/alpha ratio, (xvii) alpha/theta ratio, and (xviii) aperiodic exponent; and optionally, (a) selecting, or excluding, the patient for a clinical trial based on the outcome prediction, or (b) weighting the results of each patient based on the outcome prediction (e.g., by propensity score weighting).

Another aspect is a method for selecting a patient suffering from a psychiatric disorder for a clinical trial which has a placebo or sham treatment arm (e.g., a placebo treatment arm). The method comprises: collecting brain wave activity data via EEG electrodes applied to the patient before administration of the placebo treatment or performing the sham treatment; analyzing the brain wave activity data to calculate one or more EEG measures, and predicting an outcome of the placebo or sham treatment in the patient based on the one or more EEG measures, wherein the one or more EEG measures comprise one or more of (i) delta power features (such as relative delta power features, spatially normed delta power features, or spatially normed relative delta power features), (ii) theta power features (such as relative theta power features, spatially normed theta power features, or spatially normed relative theta power features), (iii) alpha power features (such as relative alpha power features, spatially normed alpha power features, or spatially normed relative alpha power features), (iv) beta power features (such as relative beta power features, spatially normed beta power features, or spatially normed relative beta power features), (v) gamma power features (such as relative gamma power features, spatially normed gamma power features, or spatially normed relative gamma power features), (vi) cordance features in the delta, theta, alpha, beta, or gamma frequency range, (vii) sample entropy in the delta, theta, alpha, beta, or gamma frequency range, (viii) Lempel-Ziv complexity in the delta, theta, alpha, beta, or gamma frequency range, (ix) Lyapunov complexity in the delta, theta, alpha, beta, or gamma frequency range, (x) Katz fractal dimension in the delta, theta, alpha, beta, or gamma frequency range, (xi) covariance in the delta, theta, alpha, beta, or gamma frequency range, (xii) power envelope connectivity in the delta, theta, alpha, beta, or gamma frequency range, (xiii) coherence in the delta, theta, alpha, beta, or gamma frequency range, (xiv) weighted phase lag index in the theta, alpha, beta, or gamma frequency range, (xv) beta/theta ratio, (xvi) beta/alpha ratio, (xvii) alpha/theta ratio, and (xviii) aperiodic exponent; and selecting the patient for a clinical trial based on the outcome prediction.

Yet another aspect is a method of weighting the results of each patient in a clinical trial having a placebo or sham treatment arm, where each patient suffers from a psychiatric disorder. The method comprises: collecting brain wave activity data via EEG electrodes applied to each patient before administration of the placebo treatment or performing the sham treatment; analyzing the brain wave activity data to calculate one or more EEG measures, and predicting an outcome of the placebo or sham treatment in each patient based on the one or more EEG measures, wherein the one or more EEG measures comprise one or more of (i) delta power features (such as relative delta power features, spatially normed delta power features, or spatially normed relative delta power features), (ii) theta power features (such as relative theta power features, spatially normed theta power features, or spatially normed relative theta power features), (iii) alpha power features (such as relative alpha power features, spatially normed alpha power features, or spatially normed relative alpha power features), (iv) beta power features (such as relative beta power features, spatially normed beta power features, or spatially normed relative beta power features), (v) gamma power features (such as relative gamma power features, spatially normed gamma power features, or spatially normed relative gamma power features), (vi) cordance features in the delta, theta, alpha, beta, or gamma frequency range, (vii) sample entropy in the delta, theta, alpha, beta, or gamma frequency range, (viii) Lempel-Ziv complexity in the delta, theta, alpha, beta, or gamma frequency range, (ix) Lyapunov complexity in the delta, theta, alpha, beta, or gamma frequency range, (x) Katz fractal dimension in the delta, theta, alpha, beta, or gamma frequency range, (xi) covariance in the delta, theta, alpha, beta, or gamma frequency range, (xii) power envelope connectivity in the delta, theta, alpha, beta, or gamma frequency range, (xiii) coherence in the delta, theta, alpha, beta, or gamma frequency range, (xiv) weighted phase lag index in the theta, alpha, beta, or gamma frequency range, (xv) beta/theta ratio, (xvi) beta/alpha ratio, (xvii) alpha/theta ratio, and (xviii) aperiodic exponent; and weighting the results of each patient based on the outcome prediction.

Yet another aspect is a method of determining the treatment non-specific response for a patient who suffers from a psychiatric disorder and is to be treated with a therapeutic treatment (e.g., treatment with a neurological agent, such as an antidepressant). The method comprises: collecting brain wave activity data via EEG electrodes applied to each patient before initiation of the therapeutic treatment; analyzing the brain wave activity data to calculate one or more EEG measures, and predicting the treatment non-specific response in each patient based on the one or more EEG measures, wherein the one or more EEG measures comprise one or more of (i) delta power features (such as relative delta power features, spatially normed delta power features, or spatially normed relative delta power features), (ii) theta power features (such as relative theta power features, spatially normed theta power features, or spatially normed relative theta power features), (iii) alpha power features (such as relative alpha power features, spatially normed alpha power features, or spatially normed relative alpha power features), (iv) beta power features (such as relative beta power features, spatially normed beta power features, or spatially normed relative beta power features), (v) gamma power features (such as relative gamma power features, spatially normed gamma power features, or spatially normed relative gamma power features), (vi) cordance features in the delta, theta, alpha, beta, or gamma frequency range, (vii) sample entropy in the delta, theta, alpha, beta, or gamma frequency range, (viii) Lempel-Ziv complexity in the delta, theta, alpha, beta, or gamma frequency range, (ix) Lyapunov complexity in the delta, theta, alpha, beta, or gamma frequency range, (x) Katz fractal dimension in the delta, theta, alpha, beta, or gamma frequency range, (xi) covariance in the delta, theta, alpha, beta, or gamma frequency range, (xii) power envelope connectivity in the delta, theta, alpha, beta, or gamma frequency range, (xiii) coherence in the delta, theta, alpha, beta, or gamma frequency range, (xiv) weighted phase lag index in the theta, alpha, beta, or gamma frequency range, (xv) beta/theta ratio, (xvi) beta/alpha ratio, (xvii) alpha/theta ratio, and (xviii) aperiodic exponent; and optionally calculating the treatment specific response in each patient.

In one embodiment of any of the methods described herein, the EEG measures include alpha power over 8 to 12 Hz, or alternative over 7 to 13 Hz.

In one embodiment of any of the methods described herein, the EEG measures include spatially normed alpha power (e.g., spatially normed alpha (8-12 Hz) power). In another embodiment of any of the methods described herein, the EEG measures include spatially normed absolute alpha power, spatially normed relative alpha power, or both (e.g., spatially normed absolute alpha (8-12 Hz) power, spatially normed relative alpha (8-12 Hz) power, or both). In yet another embodiment of any of the methods described herein, the EEG measures include both spatially normed absolute alpha (8-12 Hz) power and spatially normed relative alpha (8-12 Hz) power.

In yet another embodiment of any of the methods described herein, the spatially normed absolute alpha power is calculated by Z-scoring log-transformed alpha power across channels, and the spatially normed relative alpha power is calculated by Z-scoring log-transformed relative alpha power across channels.

In yet another embodiment of any of the methods described herein, the alpha (8-12 Hz) power features are extracted from the brain wave activity data and spatially normed.

In yet another embodiment of any of the methods described herein, the one or more EEG measures, in addition to spatially normed alpha (8-12 Hz) power features, are measures of predictability, measures of regularity, or any combination thereof.

In yet another embodiment of any of the methods described herein, the one or more EEG measures are analyzed with stored historical subject data containing data from a plurality of subjects having a psychiatric disorder (e.g., depressive disorder), who received a therapeutic, placebo, or sham treatment, wherein the stored historical subject data include for a plurality of the subjects, the efficacy of the therapeutic, placebo, or sham treatment and one or more of the same type of EEG measures as calculated for the patient. In one embodiment, the efficacy of the treatment (e.g., placebo or sham treatment) from the stored historical subject data has been regressed (e.g., by partial least squares analysis) on the one or more EEG measures. In another embodiment, the step of analyzing the brain wave activity data to predict an outcome of the treatment in the patient comprises determining an efficacy likelihood score for the patient based on the stored historical subject data.

In yet another embodiment of any of the methods described herein, the one or more EEG measures are analyzed with stored historical subject data containing data from a plurality of subjects having a psychiatric disorder (e.g., depressive disorder), who received a placebo treatment, wherein the stored historical subject data include for a plurality of the subjects, the efficacy of the placebo treatment and one or more of the same type of EEG measures as calculated for the patient. In one embodiment, the efficacy of the treatment (e.g., placebo treatment) from the stored historical subject data has been regressed (e.g., by partial least squares analysis) on the one or more EEG measures. In another embodiment, the step of analyzing the brain wave activity data to predict an outcome of the treatment in the patient comprises determining an efficacy likelihood score for the patient based on the stored historical subject data.

In one embodiment of any of the methods described herein, the psychiatric disorder is depression. In another embodiment of any of the methods described herein, the patients suffers from major depressive disorder. In yet another embodiment of any of the methods described herein, the psychiatric disorder is major depressive disorder, the depressive phase of bipolar disorder, depressive symptoms in post-traumatic stress disorder (PTSD), negative symptoms of schizophrenia, an anxiety disorder (such as panic disorder, social anxiety disorder, or generalized anxiety disorder).

In yet another embodiment of any of the methods described herein, predicted patient response is predicted in a post-treatment percentage change in a depression rating scale (e.g., Hamilton Depression Rating Scale (HAMD) or Montgomery-Asberg Depression Rating Scale (MADRS)).

In yet another embodiment of any of the methods described herein, the brain wave activity data is collected at resting state (i.e., resting-state EEG). In one embodiment, the brain wave activity data is collected in resting eyes-open (REO) condition, resting eyes-closed (REC) condition, or both. For example, the brain wave activity data is collected in both resting eyes-open (REO) and resting eyes-closed (REC) conditions.

In yet another embodiment of any of the methods described herein, the brain wave activity data is collected via a standard 10-20 configuration of EEG electrodes applied to the patient.

In yet another embodiment of any of the methods described herein, the brain wave activity data is collected via a reduced electrode configuration applied to the patient, such as a configuration of EEG electrodes which (i) includes no more than 18 electrodes, (ii) comprises four or more electrodes from around the circumference of the head of the patient, and (iii) two or more electrodes from the midline of the head of the patient. In one embodiment, the configuration comprises (i) four or more electrodes selected from Fp1, Fp2, F7, F8, T3, T4, T5, T6, O1, and O2 and (ii) two or more electrodes selected from Fz, Cz, and Pz, according to the 10-20 system of electrode placement. In another embodiment, the configuration comprises (i) four or more electrodes selected from Fp1, Fp2, AF7, AF8, F7, F8, FT7, FT8, T7, T8, TP7, TP8, P7, P8, PO7, PO8, O1, and O2 and (ii) two or more electrodes selected from Fpz, AFz, Fz, FCz, Cz, CPz, Pz, POz, and Oz, according to the 10-10 system of electrode placement. In one embodiment, the configuration includes no more than 9 electrodes (such as no more than 8 electrodes, for example the configuration includes 6, 7, or 8 electrodes). In yet another embodiment of any of the methods described herein, the configuration is: (i) a 7-channel electrode configuration consisting of electrodes at F7-Fz-F8-Cz-P7-Pz-P8, according to the 10-10 system of electrode placement, (ii) an 8-channel electrode configuration consisting of electrodes at F7-Fz-F8-Cz-P7-Pz-P8-Oz, according to the 10-10 system of electrode placement, (iii) an 8-channel electrode configuration consisting of electrodes at F7-Fz-F8-Cz-P7-Pz-P8-Fpz, according to the 10-10 system of electrode placement, (iv) an 8-channel electrode configuration consisting of electrodes at F7-Fz-F8-Cz-P7-Pz-P8-AUX, according to the 10-10 system of electrode placement, where the auxiliary (AUX) channel is a general electrophysiology channel (allowing for EEG, electrooculogram (EOG), electromyogram (EMG), or electrocardiogram (EKG) input), or (v) a 6-channel electrode configuration consisting of electrodes at Cz-Pz-Fp1-Fp2-P7-P8, according to the 10-10 system of electrode placement. In one embodiment, the configuration may include one or more reference electrodes (such as one or two reference electrodes), for example on the earlobe or on the mastoid, in addition to the aforementioned electrode configuration (such as the 6-, 7-, or 8-channel electrode configuration). The reduced electrode configuration may incorporated into a wearable device for application to the patient to collect EEG data. For example, the device may be a cap and/or headset made out of a semi-rigid material that is fitted onto the patient’s head and includes the reduced number of electrodes of the reduced electrode configuration.

### BRIEF DESCRIPTION OF THE DRAWINGS

For a more complete understanding of the present invention, including features and advantages, reference is now made to the detailed description of the invention along with the accompanying figures:

FIG. 1 shows graphs illustrating the Hamilton Depression Rating Scale (HAMD) change compared to the model prediction (top) and the mean HAMD change over time (bottom) for the top 40%, 50%, and 60% biomarker positive patients in the placebo arm of the EMBARC trial. [The Establishing Moderators and Biosignatures of Antidepressant Response in Clinical Care (EMBARC) study is a National Institutes of Health (NIH) funded, randomized controlled trial (RCT) designed to identify objective, biological, and clinical markers (i.e., “biomarkers” or “biosignatures”) that predict how patients with Major Depressive Disorder (MDD) respond to antidepressant treatment.]

FIG. 2 shows graphs illustrating the Montgomery–Åsberg Depression Rating Scale (MADRS) change compared to the model prediction (top) and the mean MADRS change over time (bottom) for the top 40%, 50%, and 60% biomarker positive patients in the placebo arm of the ALTO-100 phase 2b RCT. [The ALTO-100 Phase 2b trial was a 6-week, double-blind, placebo-controlled study evaluating an oral, small-molecule drug designed to enhance neural plasticity, primarily targeting MDD patients with specific, memory-based cognitive biomarkers.]

FIGS. 3A–3C illustrate a multi-part table, showing the best correlation values across channels between: (i) a placebo biomarker or spatially normed alpha power features and (ii) various EEG features. The correlations were computed using data from an observational study on major depressive disorder (N = 923). Correlations were computed within the same channel and resting-state condition (REO: eyes-open resting state; REC: eyes-closed resting state). The reported values represent the median correlation across channels. Statistical significance is indicated by asterisks (*p < 0.05, **p < 0.01, ***p < 0.001).

FIGS. 4A–4C illustrate a multi-part table, showing the median correlation values across channels between: (i) a placebo biomarker or spatially normed alpha power features and (ii) various EEG features. The correlations were computed using data from an observational study on major depressive disorder (N = 923). Correlations were computed within the same channel and resting-state condition (REO: eyes-open resting state; REC: eyes-closed resting state). The reported values represent the median correlation across channels. Statistical significance is indicated by asterisks (*p < 0.05, **p < 0.01, ***p < 0.001).

### DETAILED DESCRIPTION OF THE INVENTION

It is to be understood that the figures and descriptions of the present disclosure may have been simplified to illustrate elements that are relevant for a clear understanding of the present disclosure, while eliminating, for purposes of clarity, other elements found in a typical wearable assistance device or typical method of using a wearable assistance device. Those of ordinary skill in the art will recognize that other elements may be desirable and/or required in order to implement the present disclosure. However, because such elements are well known in the art, and because they do not facilitate a better understanding of the present disclosure, a discussion of such elements is not provided herein. It is also to be understood that the drawings included herewith only provide diagrammatic representations of the presently preferred structures of the present disclosure and that structures falling within the scope of the present disclosure may include structures different than those shown in the drawings. Reference will now be made to the drawings wherein like structures are provided with like reference designations.

The terms "treat," "treatment," and "treating" in the context of the administration of a therapy to a patient refers to the reduction or inhibition of the progression and/or duration of a disease or condition, the reduction or amelioration of the severity of a disease or condition, and/or the amelioration of one or more symptoms thereof resulting from the administration of one or more therapies.

The therapeutic treatment can be the administration of a drug, such as an antidepressant, or another treatment, such as electroconvulsive therapy (ECT), transcranial magnetic stimulation (TMS), deep brain stimulation (DBS), or vagal nerve stimulation (VNS). In one embodiment, the therapeutic treatment comprises administration of a selective serotonin reuptake inhibitor (SSRI), a serotonin and norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine-dopamine reuptake inhibitor (NDRI), a monoamine oxidase inhibitor (MAOI), a tetracyclic antidepressant (TeCA), an atypical antipsychotic, or a tricyclic antidepressant (TCA).

The term “antidepressant” unless indicated otherwise includes selective serotonin reuptake inhibitors (SSRIs) (e.g., fluoxetine, escitalopram, citalopram, and sertraline), selective serotonin and norepinephrine reuptake inhibitors (SNRIs) (e.g., venlafaxine, duloxetine, and milnacipran), norepinephrine and dopamine reuptake inhibitors (e.g., bupropion), atypical antidepressants, agomelatine, and any combination of any of the foregoing. In one embodiment, the antidepressant is selected from an SSRI, SNRI, or bupropion or a pharmaceutically acceptable salt thereof. In another embodiment, the antidepressant is selected from an SSRI (other than fluvoxamine), SNRI, or bupropion or a pharmaceutically acceptable salt thereof.

The term “treatment non-specific response” (or “non-specific responses to treatment”) refers to improvements or changes in a patient's condition that are not directly caused by the specific treatment being administered, but rather by other factors like the patient's belief in the treatment, the doctor-patient relationship, or the general expectation of getting better, often associated with the “placebo effect.”

The term “treatment specific response” refers to improvements or changes in a patient's condition that are directly caused by the specific treatment being administered.

The psychiatric disorders described herein, such as major depressive disorder, bipolar disorder, bipolar I disorder, and bipolar II disorder, are intended to be as defined in the Diagnostic and Statistical Manual of Mental Disorders (DSM-5), which is hereby incorporated by reference. The severity of depression can be measured by the Montgomery-Åsberg Depression Rating Scale (MADRS), Patient Health Questionnaire-9 (PHQ-9), Clinical Global Impression - severity Scale (CGI-S), Hamilton Depression Rating Scale (HDRS), or any combination of any of the foregoing.

As used herein, the terms "subject" and "patient" are used interchangeably and refer to a human patient unless indicated otherwise. In one embodiment, the patient suffers from a psychiatric disorder, such as those described herein. In one embodiment, the patient has major depressive disorder.

EEG electrode placements or positions on the brain (also referred to herein as “channels”) are typically labeled using a letter or letters and a number. The letter of each electrode position stands for the general brain region that the electrode would cover. From front (nasion) to back (inion), each electrode letter labeling is as follows: Fp (pre-frontal or frontal pole), F (frontal), C (central line of the brain), T (temporal), P (parietal), and O (occipital). Electrode positions lying between these lines combine multiple letters, ordered from front to back. The number of the electrode gives information about the distance from the electrode to the midline of the brain. At the midline, the electrodes are labeled with a ‘z’ to represent zero. The electrode numbers increase as you move away from the midline. Odd numbers represent electrodes on the left hemisphere and even numbers represent electrodes on the right hemisphere.

The 10-20 system is an internationally recognized method that uses anatomical landmarks (i.e. nasion, inion, and preauricular points) to standardize the placement of EEG electrodes to record brain wave activity. The 10-20 system is based on the relationship between electrode location and the underlying area of the cerebral cortex while ensuring that all brain regions are covered. For the 10-20 system, the electrodes are placed at intervals of 10% or 20% of the total distance between the reference landmarks. For example, the distance from the nasion to the inion is measured, and electrodes are placed at 10% intervals along this line and 20% intervals along the line between the left and right ear. The 10-20 system allows for equal inter-electrode spacing and the electrode placements to be proportional to skull shape and size for a consistent and replicable method of recording EEG. Similarly, the 10-10 system is an internationally recognized extension of the 10-20 system nomenclature, placing electrodes in 10% intervals and therefore providing more detailed coverage of the scalp.

In one embodiment, the brain wave activity may have a frequency of, for example, delta (0.5-4 Hz), theta (5-8 Hz), alpha (8-12 Hz), beta (12-30 Hz), or gamma (30-60 Hz).

In one embodiment of any of the methods described herein, the step of weighting the results of each patient based on the outcome prediction is performed with propensity score weighting. In one embodiment, the propensity score weighting method uses target weights, which balance patient characteristics to be a match for one (target) of the two cohorts (intervention or comparator).

In another embodiment, the propensity score weighting method uses inverse propensity score weights, which balances characteristics to match the population combined from both arms. In yet another embodiment, the propensity score weighting method uses overlap weights, that estimates the treatment effect in an artificial population consisting of patients with the highest mutual overlap of propensity score between two cohorts. Any of the propensity score weighting methods described in Mlcoch et al., Value in Health, 2019, 22(12):1370-1377, which is hereby incorporated by reference, can be used in the weighting step of the methods described herein.

In another embodiment of any of the methods described herein, the step of weighting the results of each patient based on the outcome prediction is performed with inverse probability weighting. This method uses inverse probability weights based on the predicted placebo response. This method minimizes the impact of participants with a large predicted placebo response, minimizing bias and enhancing the precision of treatment effect estimation.

In yet another embodiment of any of the methods described herein, the step of weighting the results of each patient based on the outcome prediction is performed by the covariate adjustment approach. This method incorporates the placebo biomarker as an additional covariate in efficacy analyses to adjust for variance explained by nonspecific treatment response (i.e., placebo response). By controlling for predicted placebo response, e.g., using any of the aforementioned weighting methods based on the application of EEG-based biomarkers for placebo prediction, this approach reduces bias and improves the robustness of treatment effect estimation.

The biomarker(s) described herein (which includes spatially normed alpha (8-12 Hz) power features) can be further refined using EEG data from patients having the appropriate psychiatric disorder prior to treatment (e.g., placebo treatment) and a rating of their psychological change (such as change on a depression rating scale). Thereafter, the biomarker can be used to predict treatment non-specific response (such as a placebo effect). Exemplary machine learning techniques and processing of EEG data are described in U.S. Patent Publication No. 2021/0353224, which is hereby incorporated by reference, The EEG biomarker can be machine learning-derived, meaning machine learning algorithms are used to process and analyze the EEG data to identify specific patterns or features that serve as the biomarkers.

A likelihood score can be determined based on the biomarker(s) by methods known in the art, such as based on whether the biomarker value is above or below a predetermined positive threshold. In one embodiment, the analysis includes determining a treatment (e.g., placebo) efficacy likelihood score (e.g., z-score) for the patient based on the stored historical subject data, and then selecting a patient where the patient is predicted to be responsive to the treatment based on the likelihood score. The likelihood score may be binary (that is, either 0 or 1) or continuous (that is, any decimal value within a range of possible score values). In one embodiment, the likelihood score is bounded, for example, any value from 0.0 to 1.0.

### Determination of Biomarker

The biomarker was derived from a machine-learning approach. The biomarker values are the model predictions from a regression model trained to predict the post-treatment percentage changes in Hamilton Depression Rating Scale (HMAD) scores using baseline resting-state electroencephalography (rsEEG) features. Drawing on the expectation that placebo effects are common to all treatments, this model was trained on data from several open-label trials in major depressive disorder (MDD; N = 589), in which participants received standard FDA-approved antidepressants (N = 260), repetitive transcranial magnetic stimulation (rTMS; N = 252), or agomelatine, an antidepressant approved in Europe and Australia (N = 77). In greater detail, EEG features were age- and sex-normalized and standardized within each trial, and treatment response was defined as percent change in depression severity (e.g., HAMD-17) from baseline to endpoint. A feature pre-selection step retained only EEG features demonstrating adequate test–retest stability (i.e., having a concordance correlation coefficient, CCC > 0.55), after which a Partial Least Squares (PLS) regression model was trained and optimized via cross-validation on the pooled open-label dataset to predict treatment response. The resulting biomarker was then evaluated for out-of-sample generalizability in independent datasets, including an additional open-label investigational antidepressant trial (ALTO-100) and, notably, placebo arms from randomized controlled trials (e.g., EMBARC and a prospective validation in an ALTO-100 randomized controlled trial [RCT]), where it significantly predicted placebo-associated symptom change.

By utilizing a cross-intervention, open-label training paradigm for deriving the placebo biomarker, i.e., instead of requiring large placebo-arm EEG datasets (which are often limited), the training method disclosed herein leverages the substantially larger availability of open-label EEG datasets by combining heterogeneous active-treatment studies into a single training resource, which is explicitly guided by the insight that placebo-related/nonspecific improvement (e.g., expectancy/engagement/regression-to-the-mean and related placebo-linked processes) is a common component of response—irrespective of the specific intervention. This cross-intervention “common-factor” training yields a biomarker that transfers to double-blind placebo arms and can be operationalized to enhance detection of drug–placebo separation (e.g., by incorporating individual biomarker predictions as analysis weights that down-weight participants with high predicted placebo response, while retaining all randomized subjects), thereby increasing sensitivity to true pharmacologic effects in RCTs.

In summary, combining data from multiple open-label trials with different interventions for use as the training dataset, i.e., based on the expectation that placebo effects are common to all treatments, allows for a more effective and efficient derivation of the biomarker.

According to some embodiments, the features used by the model are the spatially normed alpha (8-12 Hz) power features extracted from baseline rsEEG recordings during both eyes-open and eyes-closed conditions, using channels from the standard 10-20 montage. It includes both spatially normed absolute and relative power features. The calculation steps for these features are described below.

For spatially normed absolute alpha power features:
(a) compute alpha (8-12 Hz) power for each channel,
(b) log transform the alpha power to make the data more normally distributed, and
(c) spatial normalization using Z-score across channels.

For spatially normed relative alpha power features:
(a) compute alpha (8-12 Hz) power and total (2-30 Hz) power for each channel,
(b) compute the relative alpha power by dividing the alpha power by the total power for each channel,
(c) log transform the relative alpha power, and
(d) spatial normalization using Z-score across channels.

Conducting spatial norming helps remove global fluctuations, highlighting regional differences in power distribution across the scalp. Features with a test-retest reliability lower than 0.55 (i.e., as assessed by concordance correlation coefficient in reference datasets) were excluded from modeling. Normative modeling was used to transform features into normed scores relative to healthy controls and to adjust for age and sex confounds. To further harmonize variations arising from different study designs and EEG amplifiers, the features were standardized within each study and for each EEG amplifier.

### Biomarker Performance

Cross-validation in the training set was used for hyperparameter tuning and initial model evaluation. The generalizability of this model was then evaluated using an additional MDD dataset: (1) an open-label trial of the investigational antidepressant ALTO-100 (NCT05117632; N = 135), (2) EMABRC (NCT01407094), which is a double-blind randomized clinical trial (RCT) comparing sertraline and placebo (N = 182), and (3) the ALTO-100 Phase 2b RCT (NCT05712187; N = 217). Biomarker performance was assessed by partial correlations, controlling for age, sex, and baseline severity. One-sided p-value was used for assessing statistical significance given the hypotheses are directional.

In the training set, the cross-validation model predictions correlated with treatment response at week 8 across all interventions (partial r = 0.09, p = 0.012). The partial r in the antidepressants, rTMS, and agomelatine treatment subgroups were 0.09, 0.09, and 0.20, respectively.

Testing the model in the open-label ALTO-100 trial showed that the model predictions were significantly correlated with HAMD change at week 8 (r = 0.15, p = 0.041). Assessing the correlation with changes in Montgomery-Asberg Depression Rating Scale (MADRS) showed similar results (r = 0.18, p = 0.019).

Further prospective validation in the EMBARC trial showed that the model predictions were significantly correlated with HAMD change at week 8 in the placebo arm (N = 99; partial r = 0.31, p < 0.001), demonstrating that the biomarker predicts actual placebo response. A significant correlation was also observed in the sertraline arm (N = 83; partial r = 0.19, p = 0.040).

Finally, we prospectively validated the model in the ALTO-100 Phase 2b RCT. We observed that, in the placebo arm (N = 111), the model predictions significantly correlated with MADRS response at week 2 (partial r = 0.29, p = 0.001), week 4 (partial r = 0.24, p = 0.006), and week 6 (partial r = 0.19, p = 0.029; no week 8 in this study). The results further demonstrated the capability of this biomarker for predicting placebo response. No significant correlations were observed in the ALTO-100 treatment arm (p > 0.05).

### Utility of the Biomarker in RCTs

The biomarker was evaluated to determine if it would enhance the detection of treatment effects in RCTs. Two approaches were explored:

Exclusion approach. Using the biomarker to exclude participants with high predicted placebo responses. (a) A fixed cutoff threshold is selected based on the distribution of the biomarker to define high predicted placebo response. (b) Participants whose predicted placebo responses being above the threshold are excluded from analysis.

Weighting approach. Incorporating the biomarker as propensity weights to assign lower weights to individuals with high predicted placebo response in the mixed models for repeated measures (MMRM) analysis. The following steps were used to convert the biomarker values to propensity weights: (a) Winsorize biomarker values at 5^{th} and 95^{th} percentile to reduce the impact of extreme values. (b) Shift by the minimum value to make biomarker values positive. (c) Scale the shifted biomarker values to preserve the total weights from the original set and use them as propensity weights.

These two approaches were examined in the EMABRC and ALTO-100 Phase 2b RCTs. For the exclusion approach, using a cutoff excluding roughly 50% of the sample showed that it increased the EMBARC sertraline vs. placebo effect size at the primary endpoint at week 8 from 0.19 (p = 0.093) to 0.40 (p = 0.024), and increased the ALTO-100 vs. placebo effect size at the primary endpoint week 6 from 0.13 (p = 0.231) to 0.42 (p = 0.048). For the weighting approach, the effect size rose from 0.19 (p = 0.093) to 0.26 (p = 0.036) in EMBARC and from 0.13 (p = 0.231) to 0.29 (p = 0.051) in ALTO-100 Phase 2b RCTs at their primary endpoints.

These results demonstrated that this biomarker could be used to enhance the detection of treatment effects in RCTs via accounting for individual differences in predicted placebo response.

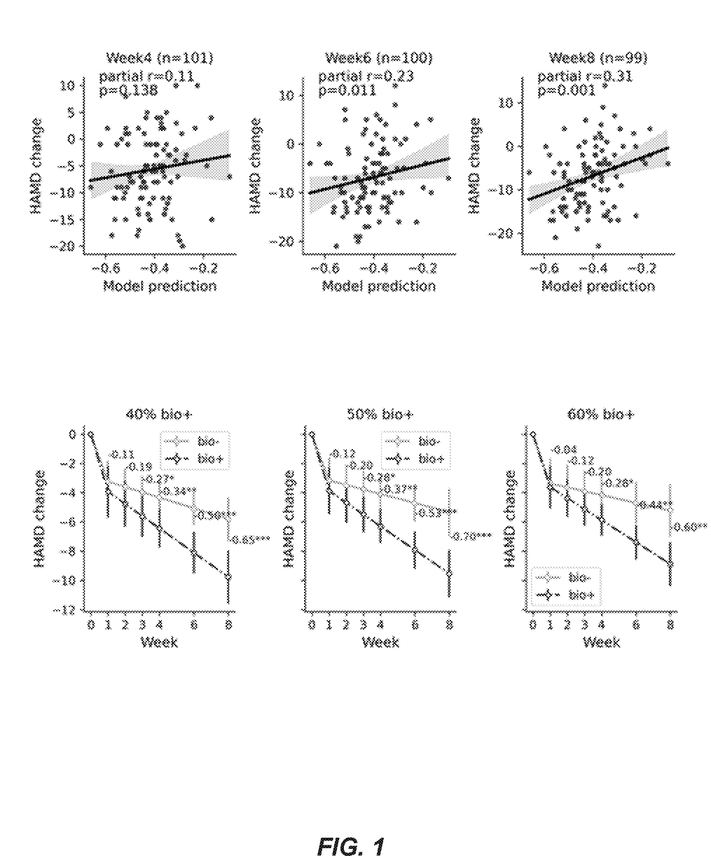

Turning now to FIG. 1, the top half of FIG. 1 illustrates the relationship between model prediction and change in HAMD from baseline at Weeks 4, 6, and 8. The solid black line shows the fitted regression with 95% confidence interval. The bottom half of FIG. 1 illustrates Longitudinal HAMD change (mean ± SE) for biomarker-positive (bio+) versus biomarker-negative (bio−) participants, where bio+ is defined by the top 40%, 50%, or 60% percentile of prediction scores. The numbers above each point indicate the Cohen’s d effect sizes for bio+ vs bio− at each week.

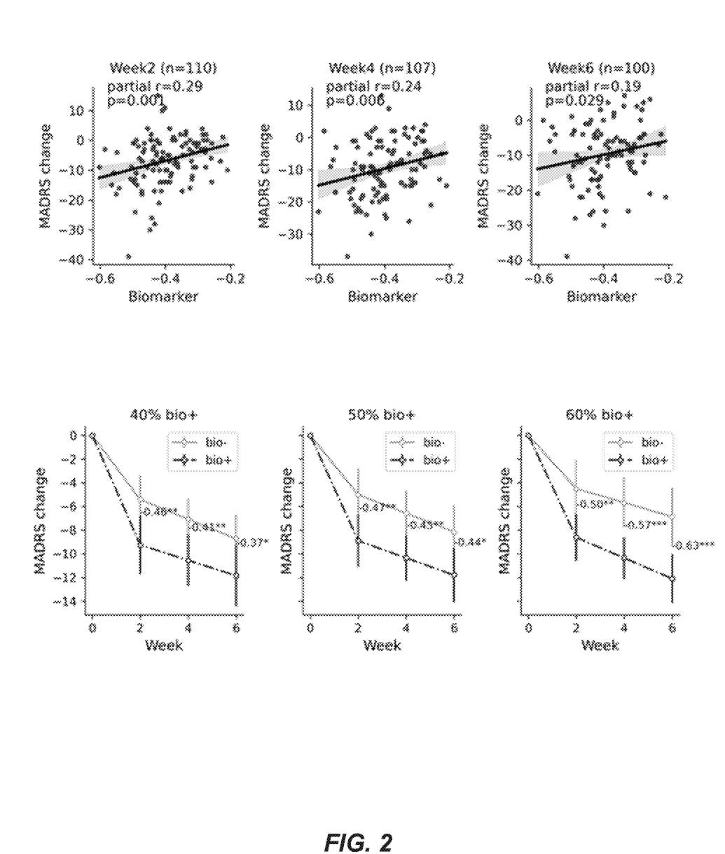

Turning next to FIG. 2, the top half of FIG. 2 illustrates the relationship between model prediction and change in Montgomery–Åsberg Depression Rating Scale (MADRS) from baseline at Weeks 2, 4, and 6. The solid black line shows the fitted regression with 95% confidence interval. The bottom half of FIG. 2 illustrates Longitudinal MADRS change (mean ± SE) for biomarker-positive (bio+) versus biomarker-negative (bio−) participants, where bio+ is defined by the top 40%, 50%, or 60% percentile of prediction scores. The numbers above each point again indicate the Cohen’s d effect sizes for bio+ vs bio− at each week.

### Other Biomarkers

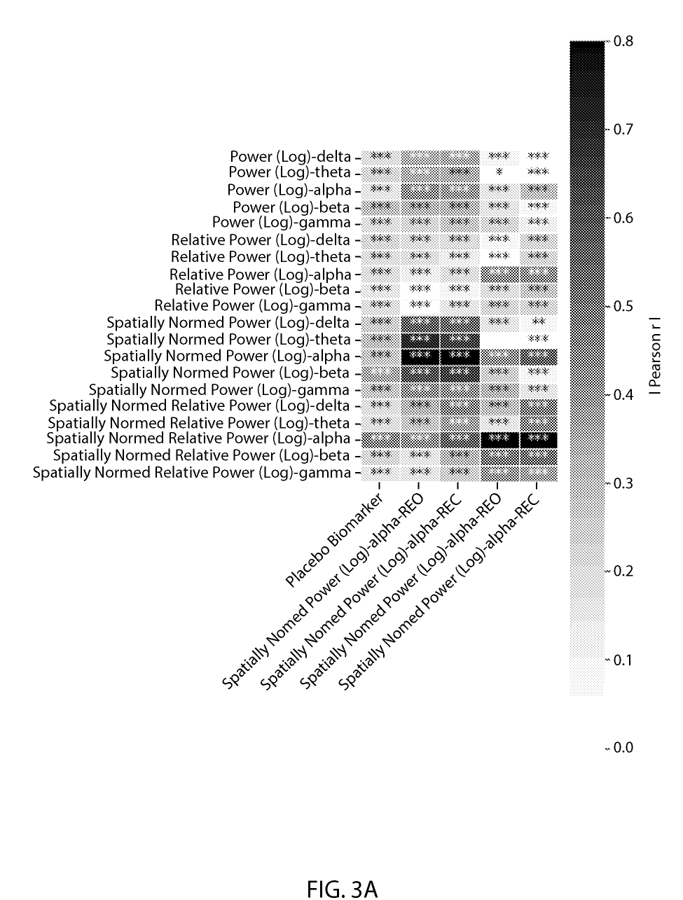

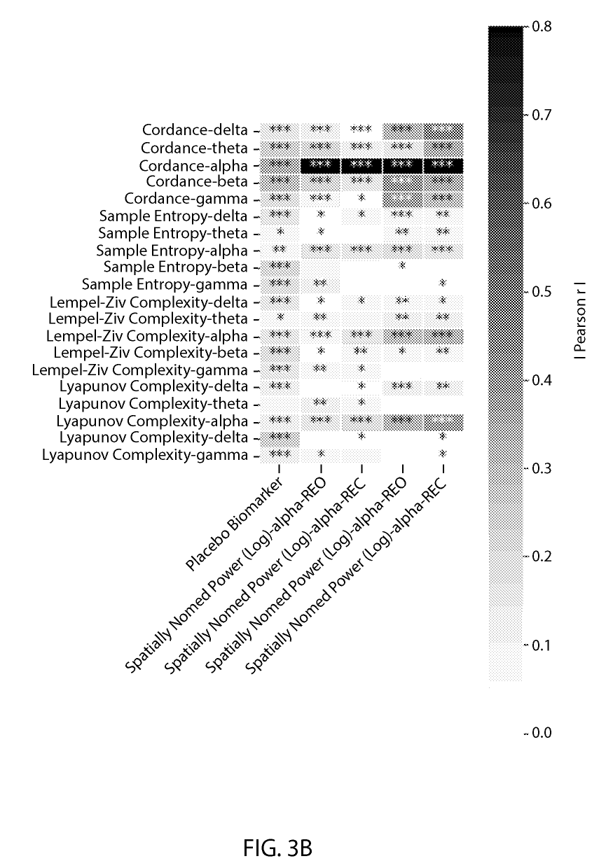

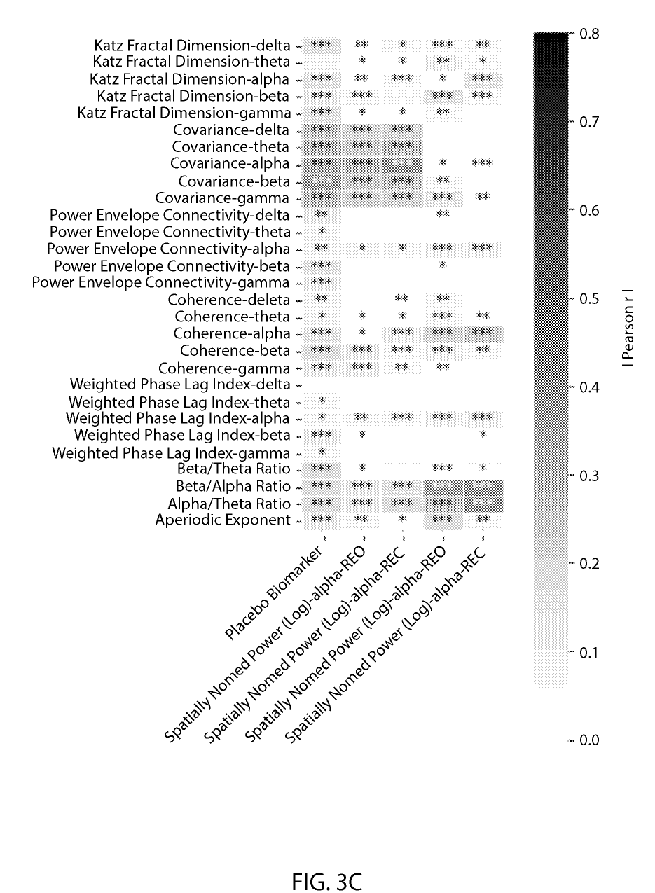

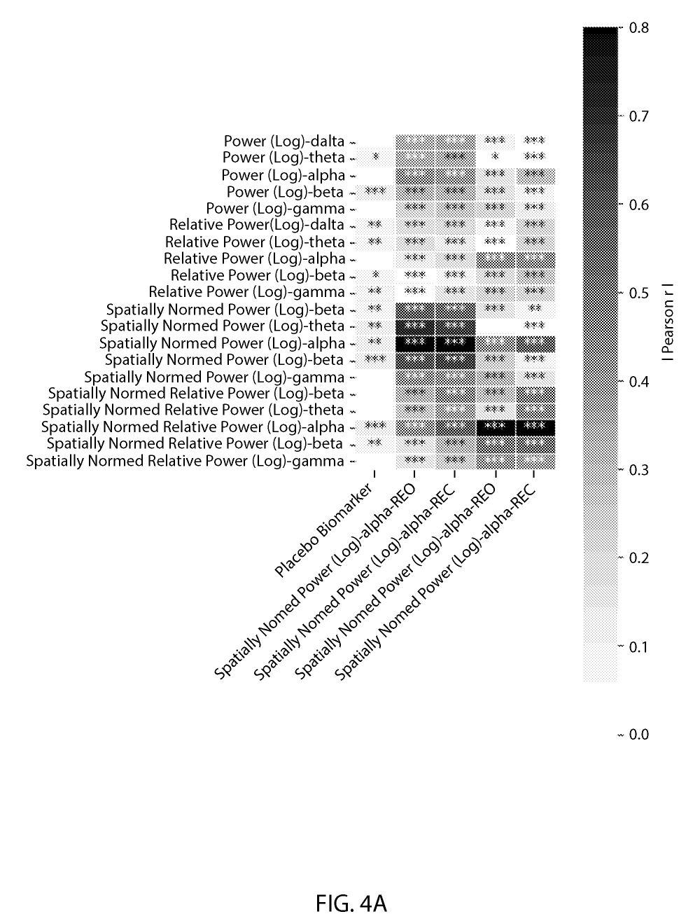

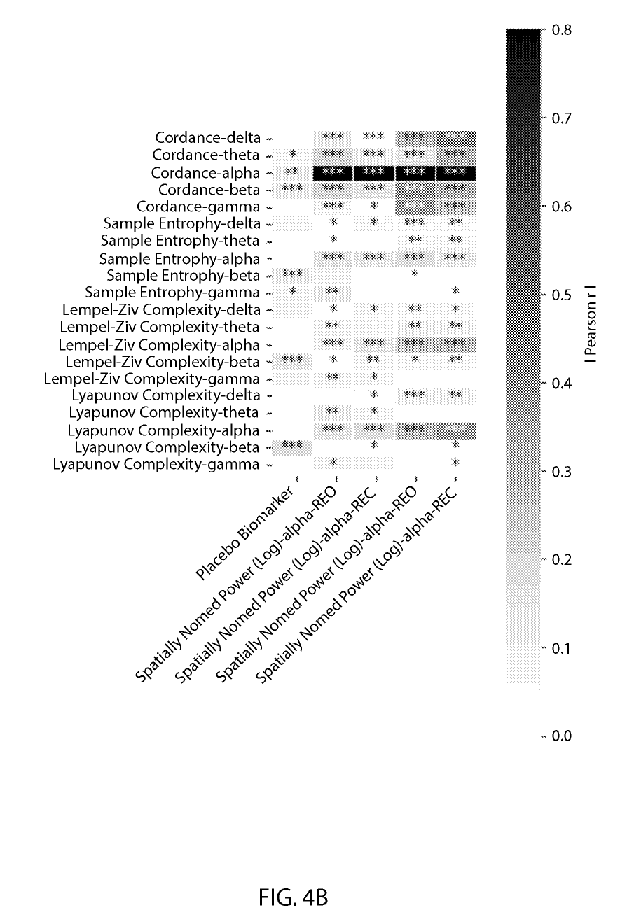

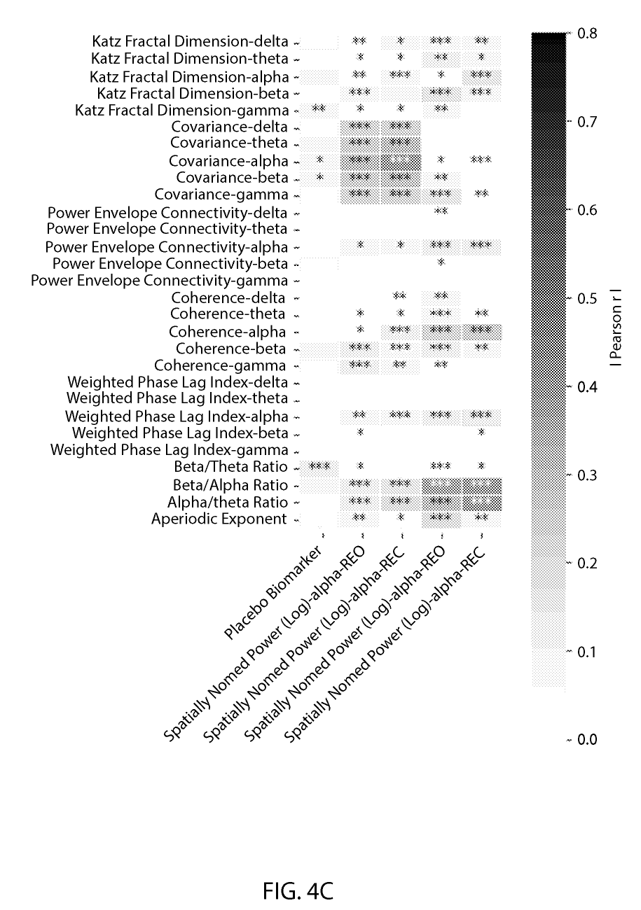

Correlations between (a) various EEG features and (b) the placebo biomarker, spatially normed alpha power (REO or REC), or spatially normed relative alpha power (REO or REC) were computed with data from an observational study on major depressive disorder (N = 923). FIGS. 3A–3C show the best correlation values across channels, while FIGS. 4A–4C show the median correlation values across channels.

Turning first to FIGS. 3A–3C, the best correlation values were determined across channels between: (i) a placebo biomarker or spatially normed alpha power features and (ii) various EEG features. The correlations were computed using data from an observational study on major depressive disorder (N = 923). Correlations were computed within the same channel and resting-state condition (REO: eyes-open resting state; REC: eyes-closed resting state). The reported values represent the best correlation across channels. Pearson r values are shown by shading (wherein darker shading represents higher Pearson r values). Statistical significance is indicated by asterisks (wherein ‘*’ means p < 0.05, ‘**’ means p < 0.01, and ‘***’ means p < 0.001). The first (i.e., leftmost) column presents the correlation between the placebo biomarker and various EEG features, with the r-values indicated in the shading representing the best correlation across channels. The remaining columns display the correlations between: spatially normed alpha power features (REO); spatially normed alpha power features (REC); spatially normed relative alpha power features (REO); and spatially normed relative alpha power features (REC).

The first part of the table, shown in FIG. 3A, depicts results for EEG features from “Power (Log) delta” to “Spatially Normed Relative Power (Log)-gamma.” The second part of the table, shown in FIG. 3B, depicts results for EEG features from “Cordance-delta” to “Lyapunov Complexity-gamma.” Finally, the third part of the table, shown in FIG. 3C, depicts results for EEG features from “Katz Fractal Dimension-delta” to “Aperiodic Exponent.”

Turning now to FIGS. 4A–4C, the median correlation values were determined across channels between: (i) a placebo biomarker or spatially normed alpha power features and (ii) various EEG features. The correlations were computed using data from an observational study on major depressive disorder (N = 923). Correlations were computed within the same channel and resting-state condition (REO: eyes-open resting state; REC: eyes-closed resting state). The reported values represent the median correlation across channels. Pearson r values are shown by shading (wherein darker shading represents higher Pearson r values). Statistical significance is indicated by asterisks (wherein ‘*’ means p < 0.05, ‘**’ means p < 0.01, and ‘***’ means p < 0.001). The first (i.e., leftmost) column presents the correlation between the placebo biomarker and various EEG features, with the r-values indicated in the shading representing the median correlation across channels. The remaining columns display the correlations between: spatially normed alpha power features (REO); spatially normed alpha power features (REC); spatially normed relative alpha power features (REO); and spatially normed relative alpha power features (REC).

The first part of the table, shown in FIG. 4A, depicts results for EEG features from “Power (Log) delta” to “Spatially Normed Relative Power (Log)-gamma.” The second part of the table, shown in FIG. 4B, depicts results for EEG features from “Cordance-delta” to “Lyapunov Complexity-gamma.” Finally, the third part of the table, shown in FIG. 4C, depicts results for EEG features from “Katz Fractal Dimension-delta” to “Aperiodic Exponent.”

As may now be appreciated, FIGS. 3A–3C and 4A–4C together show that the placebo biomarker’s strongest and most reliable relationships are with alpha/topography-derived features (with “Cordance-alpha,” shown in FIG. 3B and FIG. 4B, being a notable correlate), where FIGS. 3A–3C highlight the best single-channel “ceiling” association, and FIGS. 4A–4C confirm robustness across channels via the “median” correlation.

It will be apparent to those skilled in the art having the benefit of the teachings presented in the foregoing descriptions and the associated drawings that modifications, combinations, sub-combinations, and variations can be made without departing from the spirit or scope of this disclosure. Likewise, the various examples described may be used individually or in combination with other examples. Those skilled in the art will appreciate various combinations of examples not specifically described or illustrated herein that are still within the scope of this disclosure. In this respect, it is to be understood that the disclosure is not limited to the specific examples set forth and the examples of the disclosure are intended to be illustrative, not limiting.

As used in this specification and the appended claims, the singular forms “a”, “an” and “the” include plural referents, unless the context clearly dictates otherwise. Similarly, the adjective “another,” when used to introduce an element, is intended to mean one or more elements. The terms “comprising,” “including,” “having” and similar terms are intended to be inclusive such that there may be additional elements other than the listed elements.

Additionally, the methods described above or the method claims below do not explicitly require an order to be followed by its steps or an order is otherwise not required based on the description or claim language, it is not intended that any particular order be inferred. Likewise, where a method claim below does not explicitly recite a step mentioned in the description above, it should not be assumed that the step is required by the claim.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

1. A method for predicting the response of a patient to a placebo or sham treatment, where the patient suffers from a psychiatric disorder, the method comprising:
collecting brain wave activity data via EEG electrodes applied to the patient before administration of the placebo treatment or performing the sham treatment;
analyzing the brain wave activity data to calculate one or more EEG measures and predicting an outcome of the placebo or sham treatment in the patient based on the one or more EEG measures; and
weighting clinical trial results of the patient based on the outcome prediction of the placebo or sham treatment in the patient.

2. The method of claim 1, wherein predicting the outcome of the placebo or sham treatment in the patient based on the one or more EEG measures further comprises applying a machine-learning (ML) model to the one or more EEG measures.

3. The method of claim 1, wherein weighting the clinical trial results of the patient further comprises: down-weighting the clinical trial results of the patient in accordance with a magnitude of the outcome prediction of the placebo or sham treatment in the patient.

4. The method of claim 1, wherein the one or more EEG measures comprise one or more of:
(i) delta power features,
(ii) theta power features,
(iii) alpha power features,
(iv) beta power features,
(v) gamma power features,
(vi) cordance features in the delta, theta, alpha, beta, or gamma frequency range,
(vii) sample entropy in the delta, theta, alpha, beta, or gamma frequency range,
(viii) Lempel-Ziv complexity in the delta, theta, alpha, beta, or gamma frequency range,
(ix) Lyapunov complexity in the delta, theta, alpha, beta, or gamma frequency range,
(x) Katz fractal dimension in the delta, theta, alpha, beta, or gamma frequency range,
(xi) covariance in the delta, theta, alpha, beta, or gamma frequency range,
(xii) power envelope connectivity in the delta, theta, alpha, beta, or gamma frequency range,
(xiii) coherence in the delta, theta, alpha, beta, or gamma frequency range,
(xiv) weighted phase lag index in the theta, alpha, beta, or gamma frequency range,
(xv) beta/theta ratio,
(xvi) beta/alpha ratio,
(xvii) alpha/theta ratio, and
(xviii) aperiodic exponent.

5. A method for selecting a patient suffering from a psychiatric disorder for a clinical trial which has a placebo or sham treatment arm, the method comprising:
collecting brain wave activity data via EEG electrodes applied to the patient before administration of the placebo treatment or performing a sham treatment;
analyzing the brain wave activity data to calculate one or more EEG measures, and predicting an outcome of the placebo or sham treatment in the patient based on the one or more EEG measures, wherein predicting the outcome of the placebo or sham treatment in the patient based on the one or more EEG measures further comprises applying a machine-learning (ML) model to the one or more EEG measures; and
selecting the patient for a clinical trial based on the outcome prediction of the placebo or sham treatment in the patient.

6. The method of claim 5, further comprising: weighting clinical trial results of the patient based on the outcome prediction of the placebo or sham treatment in the patient.

7. The method of claim 5, wherein the one or more EEG measures comprise one or more of :
(i) delta power features,
(ii) theta power features,
(iii) alpha power features,
(iv) beta power features,
(v) gamma power features,
(vi) cordance features in the delta, theta, alpha, beta, or gamma frequency range,
(vii) sample entropy in the delta, theta, alpha, beta, or gamma frequency range,
(viii) Lempel-Ziv complexity in the delta, theta, alpha, beta, or gamma frequency range,
(ix) Lyapunov complexity in the delta, theta, alpha, beta, or gamma frequency range,
(x) Katz fractal dimension in the delta, theta, alpha, beta, or gamma frequency range,
(xi) covariance in the delta, theta, alpha, beta, or gamma frequency range,
(xii) power envelope connectivity in the delta, theta, alpha, beta, or gamma frequency range,
(xiii) coherence in the delta, theta, alpha, beta, or gamma frequency range,
(xiv) weighted phase lag index in the theta, alpha, beta, or gamma frequency range,
(xv) beta/theta ratio,
(xvi) beta/alpha ratio,
(xvii) alpha/theta ratio, and
(xviii) aperiodic exponent.

8. A method of determining a treatment non-specific response for a patient who suffers from a psychiatric disorder and is to be treated with a therapeutic treatment, the method comprising:
collecting brain wave activity data via EEG electrodes applied to the patient before initiation of the therapeutic treatment; and
analyzing the brain wave activity data to calculate one or more EEG measures, and predicting the treatment non-specific response in each patient based on the one or more EEG measures, wherein predicting the treatment non-specific response in the patient based on the one or more EEG measures further comprises applying a machine-learning (ML) model to the one or more EEG measures.

9. The method of claim 8, further comprising: calculating the treatment specific response in the patient.

10. The method of claim 8, wherein the one or more EEG measures include spatially normed absolute alpha (8-12 Hz) power, spatially normed relative alpha (8-12 Hz) power, or both.

11. The method of claim 10, wherein the spatially normed absolute alpha (8-12 Hz) power is calculated by Z-scoring log-transformed alpha power across channels, and the spatially normed relative alpha (8-12 Hz) power is calculated by Z-scoring log-transformed alpha power across channels.

12. The method of claim 8, wherein the one or more EEG measures comprise measures of predictability, measures of regularity, or any combination thereof.

13. The method of claim 8, wherein the one or more EEG measures are analyzed with stored historical subject data containing data from a plurality of subjects having a depressive disorder and who received a placebo treatment, and wherein the stored historical subject data includes, for a plurality of the subjects, the efficacy of the placebo treatment and one or more of the same type of EEG measures as calculated for the patient.

14. The method of claim 13, wherein the efficacy of the placebo treatment from the stored historical subject data has been regressed on the one or more EEG measures.

15. The method of claim 13, wherein the step of analyzing the brain wave activity data further comprises determining a placebo efficacy likelihood score for the patient based on the stored historical subject data.

16. The method of claim 1, wherein the brain wave activity data is collected in resting eyes-open (REO) condition, resting eyes-closed (REC) condition, or both.

17. The method of claim 8, wherein the brain wave activity data is collected via a configuration of EEG electrodes applied to the patient, where the configuration (i) includes no more than 10 electrodes, (ii) comprises four or more electrodes from around the circumference of the head of the patient, and (iii) two or more electrodes from the midline of the head of the patient.

18. The method of claim 17, wherein the configuration comprises (i) four or more electrodes selected from Fp1, Fp2, F7, F8, T3, T4, T5, T6, O1, and O2 and (ii) two or more electrodes selected from Fz, Cz, and Pz, according to the 10-20 system of electrode placement.

19. The method of claim 17, wherein the configuration comprises (i) four or more electrodes selected from Fp1, Fp2, AF7, AF8, F7, F8, FT7, FT8, T7, T8, TP7, TP8, P7, P8, PO7, PO8, O1, and O2 and (ii) two or more electrodes selected from Fpz, AFz, Fz, FCz, Cz, CPz, Pz, POz, and Oz, according to the 10-10 system of electrode placement.

20. The method of claim 17, wherein the configuration is one of:
(a) a 7-channel electrode configuration consisting of electrodes at F7-Fz-F8-Cz-P7-Pz-P8, according to the 10-10 system of electrode placement,
(b) an 8-channel electrode configuration consisting of electrodes at F7-Fz-F8-Cz-P7-Pz-P8-Oz, according to the 10-10 system of electrode placement,
(c) an 8-channel electrode configuration consisting of electrodes at F7-Fz-F8-Cz-P7-Pz-P8-Fpz, according to the 10-10 system of electrode placement,
(d) an 8-channel electrode configuration consisting of electrodes at F7-Fz-F8-Cz-P7-Pz-P8-AUX, according to the 10-10 system of electrode placement, where the auxiliary (AUX) channel is a general electrophysiology channel (allowing for EEG, electrooculogram (EOG), electromyogram (EMG), or electrocardiogram (EKG) input), or
(e) a 6-channel electrode configuration consisting of electrodes at Cz-Pz-Fp1-Fp2-P7-P8, according to the 10-10 system of electrode placement.
